(19) United States
(12) Patent Application Publication
(10) Pub. No.: US 2010/0137674 A1
(43) Pub. Date: Jun. 3, 2010

(54) BRACHYTHERAPY TREATMENT OF THE SPINE WITH IRRADIATED IMPLANTS

(76) Inventors: David Evans, Downingtown, PA (US); William Woodburn, Mantua, NJ (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/626,010

(22) Filed: Nov. 25, 2009

Related U.S. Application Data
(60) Provisional application No. 61/118,235, filed on Nov. 26, 2008.

(51) Int. Cl. A61M 36/12

(52) U.S. Cl. 600/7

(57) ABSTRACT

A spinal implant configured to facilitate focused treatment of spinal tumors includes a plurality of irradiated implant seeds configured to concentrate the dosage and duration of radiation treatment of tumors.

### CROSS REFERENCE TO RELATED APPLICATIONS

This application claims the benefit of U.S. Provisional Patent Application Ser. No. 61/118,235 filed Nov. 26, 2008, the disclosure of which is hereby incorporated by reference as if set forth in its entirety herein.

### BACKGROUND

The treatment of spinal tumors typically involves removal of healthy tissue that surrounds the tumor in order to avoid violating the tumor outer capsule, which can allow malignant cells to escape and metastasize. In some instances, removal of the tumor involves violation of the tumor outer capsule. For example, where the tumor surrounds the spinal cord, the tumorous tissue is cut in order to allow the tumor to slide past the spinal cord during removal. Regardless of whether the outer capsule of the tumor is violated during removal of the tumor, it is usually desirable to apply radiation therapy to the tumor location in order to reduce the risk of metastasis. Radiation therapy is also applied in instances where the tumor is not removed, either because the tumor is inoperable or because the surgical procedure would be impractical.

Radiation therapy is conventionally delivered to the spinal region using an extracorporeal radiation source, such as a proton beam, that is directed generally at the tumor or incision site, or can alternatively be delivered more systemically using intravenous delivery. Unfortunately, conventional spinal radiation therapy exposes a large quantity of healthy tissue to radiation.

Brachytherapy has been used to treat prostate cancer interstitially by implanting seeds having concentrated doses of radiation within the prostate tissue. Low dose radiation (LDR) seeds are implanted surgically into the prostate, and remain in the prostate permanently. High dose radiation (HRD) seeds are implanted in the prostate on a more temporary basis.

Conventional LDR seeds include casings sized as desired and formed from an implant grade material, such as titanium, and filled with a radioactive material. The seeds can be provided as stranded seeds whose casings are attached to one or more other pellets, or free seeds whose pellets are not attached to any other casings. The casings can be filled with any suitable isotope, depending on the desired level of radiation emission and half life. Typical isotopes include iodine-125 and palladium-103. Both materials continuously emit low-energy x-rays that travel only a short distance, which keeps the radiation away from surrounding organs.

HDR brachytherapy seeds involve the temporary insertion of wire made from iridium-195, which emits a higher-energy x-ray than palladium or iodine. Typically, thin plastic catheters are inserted into the prostate through a template. The iridium wire is inserted into these catheters one at a time then left in place for a short period of time, for instance a few seconds.

It is therefore desirable to provide a focused irradiated implant for the treatment of spinal tumors.

### SUMMARY

In accordance with one aspect, a brachytherapy spinal implant is provided, including a spinal implant and a plurality of irradiated seeds. The spinal implant includes a body extending along a longitudinal axis, a superior endplate disposed at one longitudinal end of the body, and an inferior endplate disposed at an opposing longitudinal end of the body, each end plate defining a vertebral-engaging surface configured to engage a complementary vertebral endplate. The irradiated seeds are attached to the implant body so as to direct radiation toward the spine in a desired direction when the brachytherapy implant is disposed in an intervertebral space.

### BRIEF DESCRIPTION OF THE DRAWINGS

The foregoing summary, as well as the following detailed description of a preferred embodiment of the application, will be better understood when read in conjunction with the appended drawings. For the purposes of illustrating the irradiated spinal implant for brachytherapy treatment of the present application, there is shown in the drawings a preferred embodiment. It should be understood, however, that the application is not limited to the precise arrangements and instrumentalities shown. In the drawings:

FIG. 1 is a top perspective view of a brachytherapy spinal implant constructed in accordance with one embodiment including a brachytherapy seeds attached to a spinal implant body;

FIG. 2A is a side elevation view of one of the brachytherapy seeds illustrated in FIG. 1;

FIG. 2B is a sectional perspective view of one of the brachytherapy seeds illustrated in FIG. 1;

FIG. 3A is a top plan view of the brachytherapy spinal implant illustrated in FIG. 1 showing an endplate thereof;

FIG. 3B is an enlarged top plan view of the endplate illustrated in FIG. 3A, showing seed-receiving apertures;

FIG. 3C is a top plan view similar to FIG. 3B, showing the brachytherapy seeds installed in the seed-receiving apertures;

FIG. 3D is a sectional side elevation view of a portion of the endplate illustrated in FIG. 3C constructed in accordance with one embodiment;

FIG. 3E is a perspective view of a portion of the endplate illustrated in FIG. 3A as indicated at line 3E-3E of FIG. 1, showing one of the seeds being inserted into the endplate;

FIG. 4A is a perspective view of one of the brachytherapy seeds illustrated in FIG. 1 constructed in accordance with one embodiment;

FIG. 4B is a perspective view of one of the brachytherapy seeds illustrated in FIG. 1 attached to the implant in accordance with another embodiment;

FIG. 4C is an exploded perspective view of one of the brachytherapy seeds illustrated in FIG. 1 attached to the implant in accordance with another embodiment;

FIG. 4D is a schematic exploded top plan view of a portion of the implant illustrated in FIG. 1, showing one of the seeds attached to the implant in accordance with another embodiment;

FIG. 4E is a schematic exploded to plan view of a portion of the implant illustrated in FIG. 1, showing one of the seeds attached to the implant in accordance with another embodiment;

FIG. 4F is an exploded perspective view of a portion of the implant illustrated in FIG. 1, showing one of the seeds attached to the implant in accordance with another embodiment;

FIG. 5A is a perspective view of a brachytherapy bone fastener constructed as a bone screw in accordance with one embodiment;

FIG. 5B is a sectional side elevation view of the bone screw illustrated in FIG. 5A, illustrating an embedded brachytherapy seed;

FIG. 5C is a sectional side elevation view of the bone screw illustrated in FIG. 5A, illustrating a pair of embedded brachytherapy seeds;

FIG. 5D is a sectional side elevation view of the bone screw illustrated in FIG. 5A, illustrating a plurality of embedded brachytherapy seeds;

FIG. 5E is a sectional side elevation view of a brachytherapy bone fastener containing a radionuclide in accordance with another embodiment;

FIG. 5F is a sectional side elevation view of a brachytherapy bone fastener constructed from a radionuclide in accordance with another embodiment;

FIG. 5G is a sectional side elevation view of a brachytherapy bone fastener illustrated as a bone nail;

FIG. 6A is a perspective view of a portion of a human spine having a malignant vertebral body;

FIG. 6B is a perspective view similar to FIG. 6A, showing a plurality of brachytherapy seeds implanted in and around the spine;

FIG. 6C is a perspective view of the portion of the spine illustrated in FIG. 6A with the malignant vertebral body removed, and brachytherapy seeds implanted in and around the spine; and

FIG. 6D is a perspective view of similar to FIG. 6C, but showing an intervertebral implant disposed between adjacent vertebrae occupying an intervertebral space where the malignant vertebral body was removed.

### DETAILED DESCRIPTION

Certain terminology is used in the following description for convenience only and is not limiting. The words “right”, “left”, “lower” and “upper” designate directions in the drawings to which reference is made, it being appreciated that the actual orientation may differ during use. The directional terms “inner” and “outer” refer to directions toward and away from, respectively, the geometric center of the brachytherapy implant 20 and related parts thereof, unless otherwise indicated. The words, “distal,” “proximal,” “anterior”, “posterior”, “superior,” “inferior,” “medial,” and “lateral” and related words and/or phrases designate preferred positions and orientations in the human body to which reference is made and are not meant to be limiting. The terminology includes the above-listed words, derivatives thereof and words of similar import.

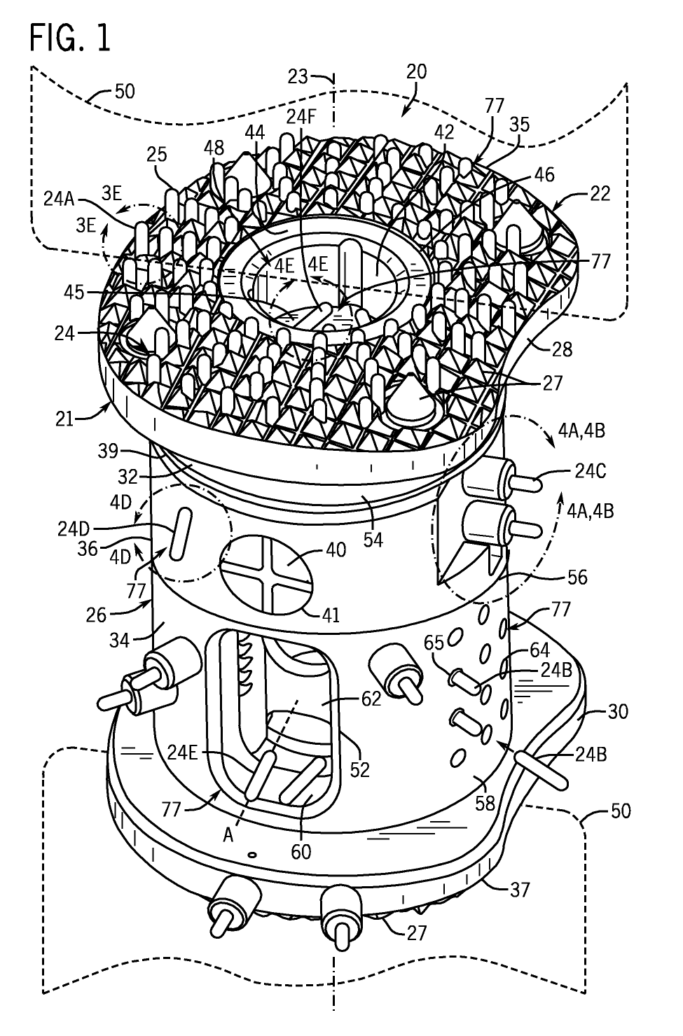

Referring to FIG. 1, a brachytherapy spinal implant 20 is configured as a spinal implant 21, which is illustrated in accordance with one embodiment as an expandable intervertebral implant 22, that retains or otherwise carries or supports one or more radiation sources illustrated irradiated seeds 24 at one or more seed attachment locations 77 of the implant 22. The irradiated seeds 24 are configured to emit predetermined doses of radiation. The implant 22 is illustrated as an expandable corpectomy spacer configured to replace at least a portion of a diseased or damaged vertebral body. The implant 22 includes superior and inferior endplates 28 and 30, respectively, sized and configured to contact corresponding neighboring engagement surfaces of neighboring vertebrae 50 and can be provided in a range of surface geometries to accommodate a range of lordotic or kyphotic angles defined by the neighboring vertebrae 50. In one embodiment, the engagement surfaces of the neighboring vertebrae 50 define vertebral endplates. Alternatively, for instance when a hemicorpectomy is performed, the engagement surfaces of one or both of the neighboring vertebrae 50 can be defined by a sectioned vertebra. The superior and inferior endplates 28 and 30 thus present respective vertebral-engaging surfaces 35 and 37 that carry outwardly projecting anti-expulsion features such as teeth, spikes, ridges, or other texturing 27. The endplates 28 and 30 are disposed at opposing ends of an implant body 26 that extends along a central longitudinal axis 23.

The implant body 26 includes a first or inner body member 32 having a generally tubular configuration, a second or outer body member 34 having a generally tubular configuration, and an expansion ring 36, all of which are hollow so as to provide an axial bore interior to and along a longitudinal axis 23 of the adjustable intervertebral implant 22. The body members 32 and 34 and the expansion ring 36 each presents a respective radially outer surface 54, 56, and 58. In use, the first or inner member 32 is preferably sized and configured to be slightly smaller than the second or outer member 34 so that the first member 32 is moveably disposed within the second member 34. However it should be noted that other arrangements of moveably associating the first member 32 and the second member 34 are contemplated.

The endplates 28 and 30 may be connected to the inner and outer members 32 and 34, respectively, by any connection mechanism known in the art, including, but not limited to, interference-fit, threading, screwing, bonding, and the like. As illustrated, the implant 22 can include an endplate cap 42 that is inserted into a central opening 46 extending longitudinally through the respective superior endplate 28, and can be threadedly coupled to the inner member 32. Alternatively, the endplate cap 42 can be integral with the inner member 32. The endplate cap 42 defines a side wall 44 coupled at its lower end to a base 45 that cooperatively define an interior void 48. A surgeon is thus able to insert osteobiological or other biocompatible material such as PMMA or bone cement into the void 48 to promote vertebral bone fusion at the complementary end plate of the superior vertebral body 50.

In this manner, the intervertebral implant 22 can be provided in a kit with a plurality of different endplates 28 and 30, thus enabling the user to select the desired endplates that best conform with the contours of the patient's vertebral endplates. By way of example, various endplates 28 and 30 may be provided that include varying shapes including, but not limited to, circular, square, rectangular, oval, kidney-shaped, etc. and/or one or more of the following characteristics: a generally wedge-shaped surface, curved surface, flat surface, etc. Alternatively, the upper and lower endplates 28 and 30 may be integrally formed with the inner and outer members 32 and 34.

The radially inner surface of the expansion ring 36 includes threading that is configured to mate with the threading 39 formed on the outer radial surface of the inner member 32, such that rotation of the expansion ring 36 causes the inner member 32 to translate or move generally linearly with respect to the outer member 34 along the longitudinal axis 23 of the adjustable intervertebral implant 22. That is, in use, the inner and outer members 32 and 34 are preferably coaxially disposed along a common longitudinal axis 23 and are preferably slidably disposed (e.g., telescopic) with respect to one another so that the axial position of the inner member 32 is adjustable with respect to the outer member 34. Rotation of the expansion ring 36 thereby causes the inner and outer members 32 and 34 to expand or contract depending on the relative direction of the rotation.

One or more set screws 40 can be inserted into a screw opening 41 of the expansion ring 36 to selectively lock the height of the adjustable intervertebral implant 22. Alternatively, the expansion ring 36 can be self-locking. In one embodiment, the set screw 40 can be positioned at a depth sufficient to enable the expansion ring 36 to be freely rotated and provide a desired height while still ensuring the secure coupling of the set screw 40 within the corresponding opening 41 due to the interference therebetween. The advancement of the set screw 40 via an appropriate instrument causes the distal end of the advanced set screw 40 to bear against the outer surface of the inner member 32 and prevent the expansion ring 36 from further rotation, thereby locking the height of the adjustable intervertebral implant 22.

The intervertebral implant 22 may be constructed from any biocompatible material or combination of any biocompatible material known in the art including, but not limited to, stainless steel, titanium, titanium alloys, ceramics, polymers including, but not limited to polytetrafluoroethylene (“PTFE”), and the like. In one embodiment, the inner and outer tubular members 32 and 34 are formed from a radiolucent material, such as, for example, a polymer or polyetheretherketone (“PEEK”), while the expansion ring 36 is preferably formed from a metal, such as, for example, titanium or stainless steel.

The implant 22 one or more relatively large bone packing apertures 52 formed in the outer surface 58 of the outer member 34. The aperture 52 defines a shelf 60 that extends radially or horizontally through the outer member 34. The implant 22 can also include an array of smaller apertures 64 that are illustrated as extend radially into or through the outer surface 58 of the outer member 34, though it should be appreciated that the apertures 64 can also extend into the inner member 32 or ring member 36 as shown in FIG. 1. The one or more bone packing apertures 52 and smaller apertures 64 provide access to an internal bore 62 extending longitudinally inside the implant body 26. It should be appreciated that the implant 22 can be fabricated with apertures 52 and 64, or that one or more apertures 52 and 64 can be created (e.g., drilled) into the implant in situ, for instance prior to or during the surgical procedure. The bore 62 can extend through one or both endplates 28 and 30.

A surgeon is thus able to insert an osteobiological or other biocompatible material such as PMMA or bone cement through one or both of the apertures 52 and 64 into the bore 62 to promote vertebral bone fusion at end plate of the inferior vertebral body 50. Gravitational forces will assist in applying the graft material direction to the end plate of the vertebral body 50. The intervertebral implant 22 is not limited to inclusion of the bone packing aperture 52 or the smaller apertures 64, and may not include apertures therein or may include additional variably shaped or located apertures, depending upon the specific application or configuration of the implant 22. The apertures 52 and 64 can further provide seed attachment locations 77, as described in more detail below.

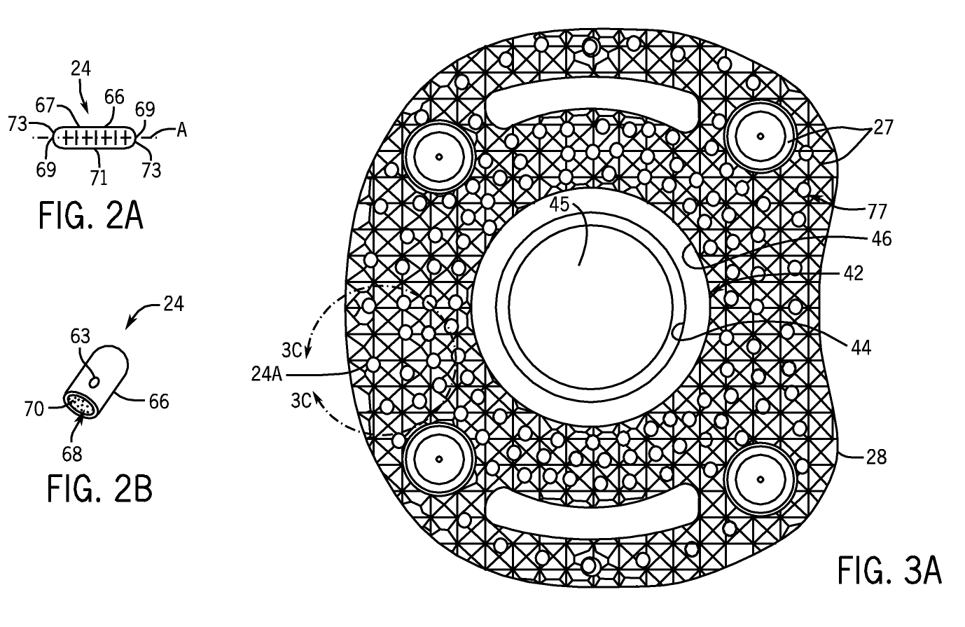

Reference now to FIGS. 1-2, the brachytherapy implant 20 includes one or more brachytherapy seeds 24 that can be mounted or otherwise attached to the intervertebral implant 22. Each brachytherapy seed 24 can include a bio-compatible protective shell 66 that defines an internal core 68 containing a radionuclide 70. The radionuclide can be selected from any suitable isotope as desired, such as an isotope of iodine, palladium, iridium, or any alternative isotope that emits a desired dose of radiation. Specific examples of isotopes include iodine-125 and palladium-103, which emit low doses of radiation, and palladium-103, which emits high doses of radiation. Thus, the radiation seeds 24 may emit radiation for a limited amount of time, e.g., a few months, but may be configured to emit radiation indefinitely or for a relatively short amount of time, depending upon treatment preferences.

The shell 66 can be constructed from any suitable implant-grade material, such as polyetheretherketone (PEEK), titanium, and the like. The shell 66 is illustrated as an elongate ovoid or tubular structure, though it can alternatively define any suitable shape as desired, such as a sphere, a curved structure, or any desired polygonal structure. As illustrated, the shell 66 defines an elongate cylindrical body 67 extending along a central axis A. The cylindrical body 57 defines a pair of opposing rounded ends 69 that define opposing outer end surfaces 73. The cylindrical body 67 defines an outer side surface 71 extending between the opposing ends 69.

In certain embodiments, it is desirable to direct radiation toward the tumor while directing the radiation away from the spinal cord. Hence, the seeds 24 can be variably positioned and oriented on the implant 22 so as to irradiate the treatment site at close range without adversely affecting other parts of the body and generally serve to prevent the growth or re-growth of malignant spinal tumors in and around the implant location. The seeds 24 can each include a radio-opaque marker 63 disposed on the shell 66 or inside the shell 66 so as to be viewable within the treatment site with the aid of fluoroscopy.

While the brachytherapy seeds 24 are illustrated and described in accordance with one embodiment, it should be appreciated that the seeds 24 could be constructed in accordance with any suitable alternative embodiment. For instance, the brachytherapy seeds 24 can define a unitary solid member constructed from a radionuclide. Furthermore, it should be appreciated that the seeds 24 can provide one or more components of the implant 22. For instance, the anti-expulsion features 27 can be formed from brachytherapy seeds 24 geometrically configured to provide teeth, spikes, ridges, texturing, or the like that projects longitudinally out from the endplates 28 and 30 as illustrated in FIG. 1.

The seeds 24 can be constructed and/or positioned as desired. The implant 22 is illustrated as including various embodiments of seeds 24 illustrated as being constructed and/or positioned as indicated by a one or more first seeds 24A, one or more second seeds 24B, one or more third seeds 24C, one or more fourth seeds 24D, one or more fifth seeds 24E, and one or more sixth seeds 24F, though it should be appreciated that the implant 22 can include any alternative type of seed connected and positioned on the implant 22 as desired.

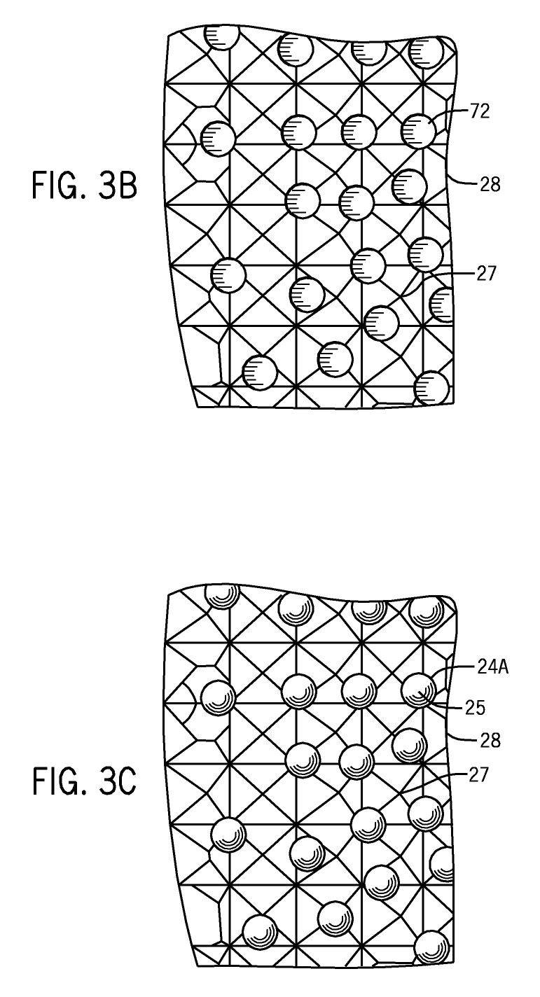

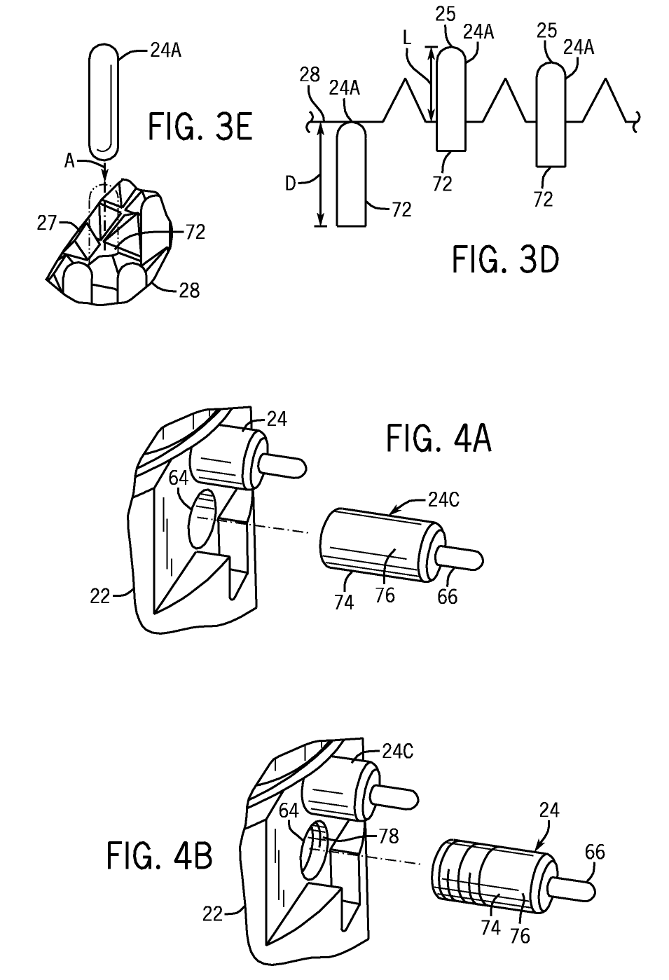

Referring also to FIGS. 3A-E, the first seeds 24A are disposed in one or both of the endplates 28 and 30 and extend longitudinally out from the endplates toward the adjacent vertebrae 50. In particular, the implant 22 can include one or more seed attachment locations 77 illustrated as seed-receiving apertures 72 extending longitudinally into one or both of the endplates 28 and 30. It should be appreciated that the implant 22 can be fabricated with apertures 72, or that one or more apertures can be created (e.g., drilled) into the implant 22 in situ, for instance prior to or during the surgical procedure.

The apertures 72 can be provided in combination with the teeth 27 as illustrated, or can be provided as an alternative to the teeth 27. One of the outer ends 69 of the first seeds 24A can be inserted into the apertures 72 in any desired manner along the direction of arrow A. For instance, the apertures 72 can define a diameter or cross-sectional dimension substantially equal to the outer diameter or alternative cross-sectional dimension of the first seeds 24A (for instance the shells 66), such that the first seeds 24A can be press-fit into the apertures 72. Alternatively or additionally, one or more of the apertures 72 can define a cross-sectional dimension greater than that of the first seeds 24A. An osteobiological or other biocompatible material such as PMMA or bone cement can be inserted into the apertures 72 to bond the shells 66 to one or both of the endplates 28 and 30. Alternatively, the shells 66 can be ultrasonically welded to the implant 22.

The apertures 72 can extend into one or both of the endplates 28 and 30 at a depth D that can be less than the longitudinal length of the seeds 24A, such that the seeds 24A are configured and positioned similar to the anti-repulsion teeth or spikes 27 common in the art for both preventing dislodgement of the brachytherapy spinal implant 20. Thus, the first seeds 24A present a vertebral-engaging surface 25 that can assist the teeth 27 of the endplates 28 and 30 in securing the implant 20 to the vertebrae 50 or may replace the teeth 27 and provide the securement of the implant 20 to the vertebrae 50. The vertebral-engaging surface 25 can be round as illustrated, or can be spiked and shaped as a pyramid or cone, as desired. Expansion of implant 20 or an alternatively constructed expandable interbody spacer can further assist in urging the surfaces 25 of the first seeds 24A to penetrate the adjacent vertebral endplates. The first seeds 24A can include additional cladding that can be coupled to the shell 66 to provide a desired geometry at the vertebral-engaging surfaces 25 configured to penetrate the adjacent vertebral endplates. The depth D of certain ones of the apertures 72 can be different than certain others of the apertures 72, such that the seeds 24A protrude at variable lengths L from one or both of the endplates 28 and 30, as desired. Alternatively, the depth D can be constant, such that the length L of protrusion is constant.

The first seeds 24A protrude superiorly from the superior endplate 28, and inferiorly from the inferior endplate 30, when the apertures 72 extend parallel to the longitudinal axis L. Alternatively, the apertures 72 can extend along a direction that intersects the longitudinal axis L, such that the axis A of the inserted seeds 24A extends in a direction having a superior or inferior directional component, and extends at an angle with respect to the longitudinal axis L.

Alternatively or additionally, one or more, up to all, of the apertures 27 can extend into one or both of the endplates 28 and 30 a depth greater than or equal to the longitudinal length of the seeds 24A, such that the seeds 24A do not protrude longitudinally out from the corresponding endplate, and are impacted in the endplate. Accordingly, some of the seeds 24A can be recessed in the endplate 28 so as to not protrude from one or both of the endplates 28 and 30, while others of the seeds 24A protrude from one or both of the endplate 28 and 30, as shown in FIG. 3D. Alternatively still, the entirety of one or both endplates 28 and 30 can be provided as a seed whose outer shell defines the outer vertebral-engaging surface of the endplates 28 and 30, and whose radionuclide is disposed inside the outer shell in the manner described above. In each of the embodiments described herein, radiation is directed outward from one or both endplates 28 and 30 in superior and inferior directions, respectively, into the adjoining vertebral bodies 50, which may be a common site for the recurrence of tumors.

Alternatively still, the seeds 24A could extend radially outward from the second body member 34, so as to provide the second seeds 24B as illustrated in FIG. 1. In particular, one of the outer ends 69 of the seeds 24B is inserted into a complementary aperture 64, which can define a diameter or cross-sectional dimension slightly greater than or substantially equal to that of the seed 24B such that the seed 24B can be press fit into the aperture 64. Alternatively, the aperture 64 can be sized greater than the seed 24C such that an osteobiological or other biocompatible material 65 such as PMMA or bone cement can be inserted into the apertures 64 to bond the shells 66 to the implant 22 inside the apertures 64. Alternatively, the shells 66 can be ultrasonically welded to the implant 22.

The aperture 64 can have a depth that causes the seed 24B to protrude out from the implant 22, or can allow the seed 24B to be recessed, or inwardly displaced, with respect to the outer wall of the implant 22. When the implant 22 is disposed between the adjacent vertebrae 50, the apertures 64 can face anteriorly, posteriorly, medially, laterally, or anywhere therebetween. Furthermore, the apertures 64 can be angled upward in a superior direction, or downward in an inferior direction. Accordingly, seeds 24B can be installed in the apertures 64 so as to direct radiation anteriorly, posteriorly, medially, laterally, or anywhere therebetween. Thus, the radiation can be directed toward tumor sites and away from healthy tissue. In one embodiment, it may be desirable to attach the radiation seeds 24 such that they do not extend in a direction toward the spinal cord.

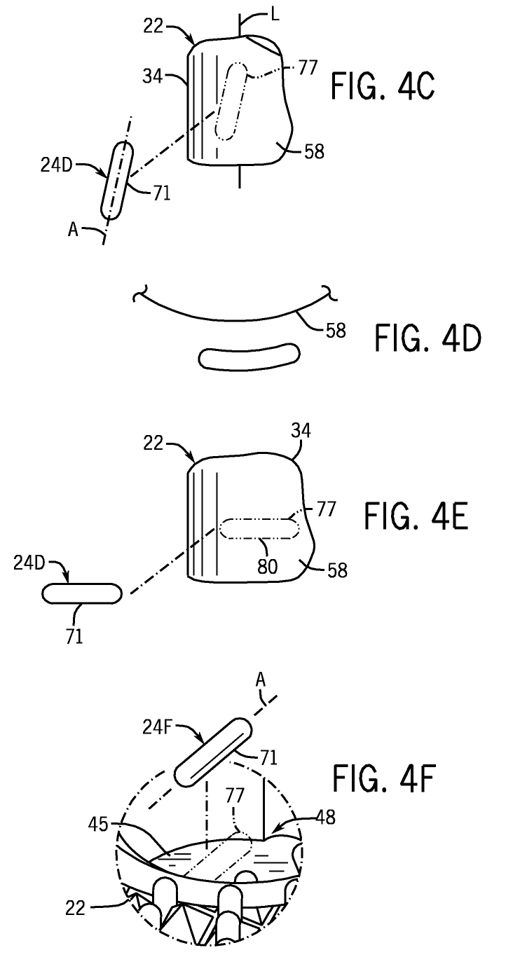

Referring now to FIGS. 1 and 4A-B, the one or more third seeds 24C include an coupling element 74 extending out from the shell 66. In one embodiment, the coupling element 74 includes cladding 76 that facilitates attachment of the seeds 24C to the implant 22. The cladding can be provided as a polymer, such as poly-ether-ether-ketone (PEEK) or titanium or any alternative implant-grade material, and can thus be made from the same material as part or all of the implant 22. The seeds 24C can be provided with the coupling element 74 attached to the shell 66, or the coupling element can be provided separately and attached to the shell in situ. In one embodiment the coupling element 74 is ultrasonically welded to the shell 66.

The coupling element 74 has a diameter or cross-sectional dimension substantially equal to or slightly less than that of the aperture 64, such that the coupling element is press-fit into the apertures 64 as illustrated in FIG. 4A. Alternatively, the coupling element 74 can define a diameter or cross-sectional dimension substantially less than that of the aperture 64, such that the shell 66 is loosely received in the aperture 64 and adhesively attached therein. The coupling element 74 can be attached to the implant 22 using a bone cement or alternative adhesive that bonds the coupling element 74 to the implant 22 in the corresponding aperture 64. Alternatively, the coupling element can be ultrasonically welded to the implant 22. Alternatively still, the coupling element 74 can have a threaded outer surface 75 configured to mate with a correspondingly threaded inner surface 78 of the apertures 64 as illustrated in FIG. 4B. The coupling element 74 can be coupled to one of the outer ends 69 of the shell 66, and extends along a portion of the length of the shell 66 less than an entire length of the shell 66 such that the body portion 67 of the shell 66 protrudes out from the coupling element 74.

As illustrated, the shell 66 is positioned in the coupling element 74 so as to be aligned with the aperture 64 when the coupling element is inserted into the aperture 64, such that the shell 66 projects radially out from the implant 22. Alternatively, the shell 66 can be positioned in the coupling element 74 such that the shell 66 extends in a direction that is angularly offset with respect to the aperture 64 when the coupling element 74 is installed in the aperture 64.

The coupling element 74 can be made from titanium, gold, or any desired material, and can provide a radiation shield that prevents or significantly limits radiation from passing therethrough. Accordingly, the seed 24C can emit radiation from locations that are not covered by the coupling element 74. Accordingly, use of the coupling element 74 to couple or assist in coupling the second seeds 24C to the implant 22 enables a surgeon to tailor the emission of radiation from the brachytherapy spinal implant 20 by permitting selection of the location of the second seeds 24C and the number of second seeds 140b that are coupled to the implant 100. Alternatively, the second seeds 140b can be preassembled to the brachytherapy spinal implant 100.

Referring now to FIGS. 4C-E, one or more fourth seeds 24D can be attached edgewise to any surface of the implant 22, such that the side surface 71 is attached to the implant 22 at seed attachment locations 77 at any surface of the inner body member 32, outer body member 34, or expansion ring 36. As illustrated, the outer surface 58 of the of the outer body member 34 defines the seed attachment location 77.

As illustrated in FIG. 4C, for instance, the fourth seeds 24D can be attached directly to the outer surface 58 of the outer body member 34. In particular, an osteobiological or other biocompatible material such as PMMA or bone cement, can be applied to the seeds 24D and/or the outer surface 58, and the side surface 71 can then be applied to the outer body member 34 at a seed attachment location 77. Alternatively, the seed 24D can be ultrasonically welded to the implant 22.

The seed 24D can be oriented such that the central axis A extends substantially parallel to the longitudinal axis so that an adequate length of the seed 24D can be bonded to the outer surface 58. Alternatively or additionally, as illustrated in FIG. 4D, one of the side surfaces 71 can be rounded to conform to the curvature of the outer surface 58 such that the seeds 24D can be attached to the outer surface 58 at any orientation relative to the longitudinal axis L. Thus, it should be appreciated that a kit can be provided including one or more spinal implants 21 and seeds 24 not attached to the implants 21, and provided as having different shapes and/or sizes configured to attach to various attachment locations 77 of the implants 21 as described above. The spinal implants 21 can alternatively or additionally be provided having seeds pre-attached.

Referring now again to FIG. 1, the one or more fifth seeds 24E can be attached to an internal surface of the implant 22. For instance, the seeds 24E can be attached to attachment locations 77 on the shelf 60 of the bone packing aperture 52. The seeds 24E can be attached with an osteobiological or other biocompatible material such as PMMA or bone cement, in addition or alternative to a groove formed in the shelf 60 in the manner described above. Alternatively or additionally, the seeds 24E can be ultrasonically welded to the implant 22. It should thus be appreciated that the axis A of the seeds 24E can be oriented in any direction perpendicular to the longitudinal axis L of the implant 22. Thus, when the implant 22 is installed in an intervertebral space, the axis A can be oriented in a horizontal plane perpendicular to the longitudinal axis L, and in or angled with respect to the medial, lateral, posterior, or anterior direction.

Referring now to FIGS. 1 and 4F, the implant 22 can define one or more see attachment locations 77 in the endplate cap 42. As illustrated, the sixth seeds 24F are attached to the base 45 of the endplate cap 42. The seeds 24F can be attached using an osteobiological or other biocompatible material such as PMMA or bone cement, in addition or alternative to a groove formed in the base 45 in the manner described above. Alternatively, the seeds 24F can be ultrasonically welded to the implant 22. Thus, the axis A of the sixth seeds 24F can be oriented in any direction perpendicular to the longitudinal axis L of the implant 22. Thus, when the implant 22 is installed in an intervertebral space, the axis A can be oriented in a horizontal plane perpendicular to the longitudinal axis L, and in or angled with respect to the medial, lateral, posterior, or anterior direction.

While the brachytherapy implant 20 has been described in connection with the expandable implant 22, it should be appreciated that the implant 20 could assume any construction suitable for implantation in a human spine. Furthermore, while the brachytherapy implant 20 has been described as including the six types of brachytherapy seeds 24A-F at the locations illustrated and described, it should be appreciated that the implant 20 can include one or more of the types of seeds 24A-F. Furthermore, one or more, up to all, of the seeds 24A-F can be attached at any of the attachment locations 77, or at any alternative location on the implant 22 as desired.

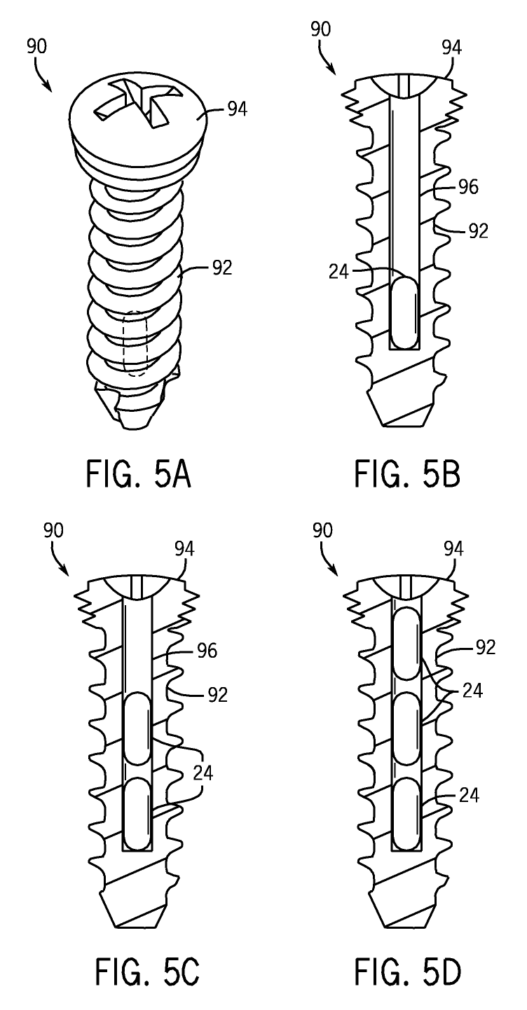

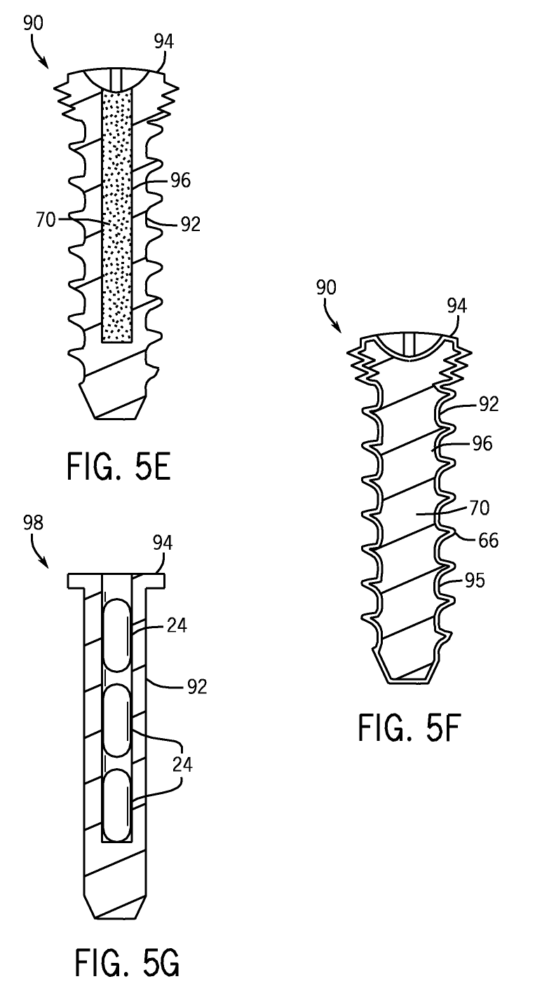

While the spinal implant 21 of the brachytherapy spinal implant 20 has been illustrated and described with respect to is configured as a spinal implant 21, which is illustrated in accordance with one embodiment as an expandable intervertebral implant 22, the spinal implant 21 can assume any alternative configuration as desired, and can be expandable or rigid. Furthermore, it should be appreciated that the implant 21 can be constructed such that the endplates 28 and 30 are pivotable with respect to each other in order to restore movement between the adjacent vertebrae. For instance, as illustrated in FIGS. 5A-D, the spinal implant 21 can be provided as a brachytherapy bone fastener such as a screw 90 that includes one or more seeds 24 mounted in or to the bone screw 210. Alternatively, the bone screw 90 can integrally contain a radionuclide. The bone screw 90 can be provided as a pedicle screw, a screw for coupling a plate to a bone, such as an anterior plate or an anterior cervical plate, or other orthopedic bone anchor.

The screw 90 can contain a longitudinally extending threaded shaft 92, and a head 94 disposed at a proximal end of the shaft 92. The screw 90 can be made from any suitable implant-grade material such as titanium. The head 94 can be threaded so as to mate with corresponding threads of a bone plate, such that the screw 90 can be a self-locking screw. The distal end of the shaft 92 can present cutting flutes such that the screw 90 is a self-tapping screw. The screw 90 can contain a cannulation 96 extending longitudinally from the head 94 into the shaft 92 that terminates proximal of the distal end of the shaft 92. The cannulation 96 can contain any number of seeds 24 as desired, for instance one, two, or three as illustrated in FIGS. 5A-D, respectively. The seeds 24 can be press-fit in the cannulation 96. Additionally or alternatively, an osteobiological or other biocompatible material such as PMMA or bone cement can be inserted into the apertures. Alternatively, the shells 66 can be ultrasonically welded to the implant 22. The one or more seeds 24 are preferably preassembled to and/or within the bone screw 90. Alternatively, the one or more seeds 24 are coupled to the bone screw 90 in situ, that is prior to surgery or intraoperatively. The bone screw 90 may be implanted into bone and the one or more seeds 24 may be subsequently coupled to the bone screw 90.

Alternatively, referring to FIG. 5E, the cannulation 96 can directly contain a radionuclide 70 of the type described above, as opposed to the cannulation 96 containing seeds 24 that contain the radionuclide 70. In this regard, it should be appreciated that the shaft 92 provides an outer shell that surrounds the radionuclide 70, such that the fastener 90 is generally constructed as described above with respect to the brachytherapy seeds 24, but is configured as a bone fastener.

Alternatively still, referring to FIG. 5F, the fastener or screw 90 can be constructed from a desired radioactive isotope of the type described above. Thus, at least one or both of the head 94 and the shaft 92 can be constructed from the isotope. A coating 95 can be applied to the outer surface of the head 94 and/or shaft 92 that is formed from the radioactive isotope, so as to provide a shell 66 of the type described above with respect to the shell 66 of the brachytherapy seeds 24.

Referring now to FIG. 5G, it should be further appreciated that the fastener 90 constructed in accordance with any of the embodiments described above could alternatively include a shaft 92 that is unthreaded, and can thus be smoothed or ribbed or otherwise textured to provide a nail or pin 98 as shown in FIG. 5G, that can be implanted directly into spinal bone or soft tissue as desired.

While examples of spinal implants 21 have been described, it should be appreciated that the brachytherapy implant 20 can include brachytherapy seeds 24 attached to or provided with any spinal implant as desired, including any desired intervertebral implant, cage, spinal rod, or the like, or other implants such as bone plates. It should be appreciated that a kit can be provided including one or more spinal implants and one or more seeds 24 that can be shaped and sized according to the method and/or location of implantation either in the spinal implant or directly into spinal bone or other tissue. Moreover, the brachytherapy seeds 24 can be constructed as an entire spinal implant, such as the screw 90, the nail 98, or a corpectomy device, an interbody spacer, a spinal rod, an implant configured for insertion interior to a vertebral body, and the like.

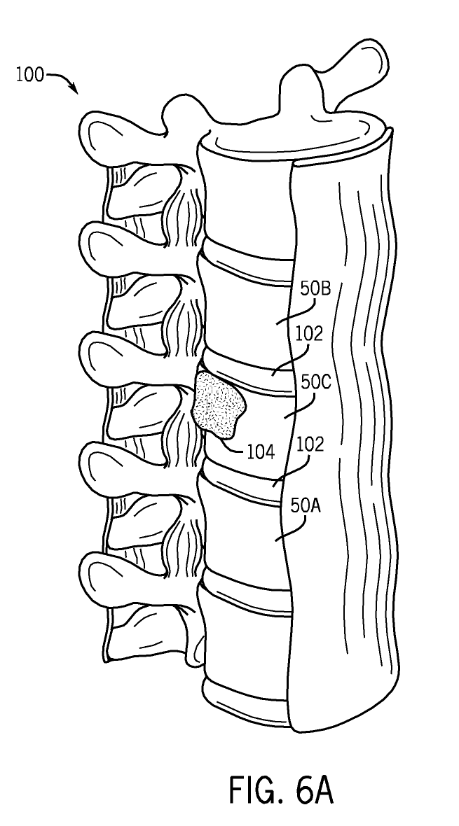

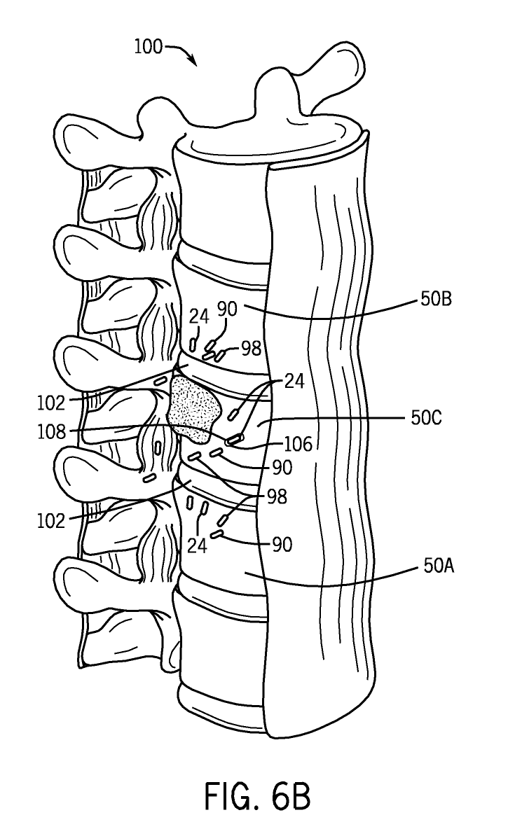

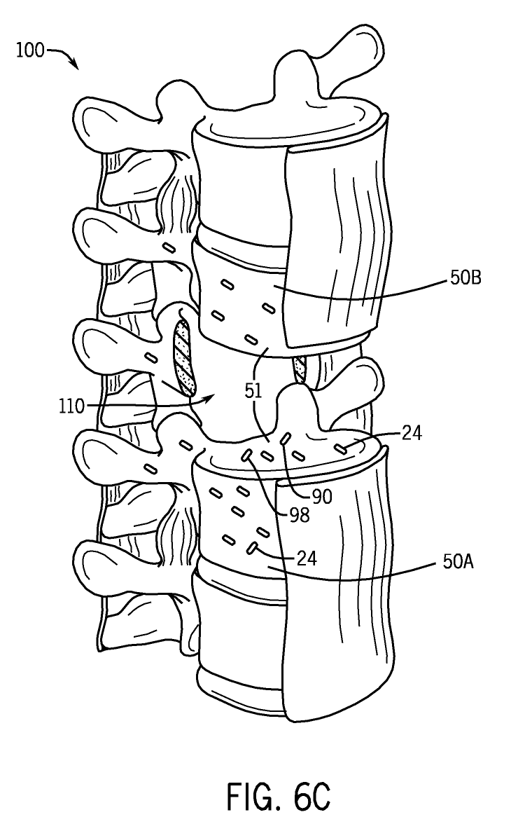

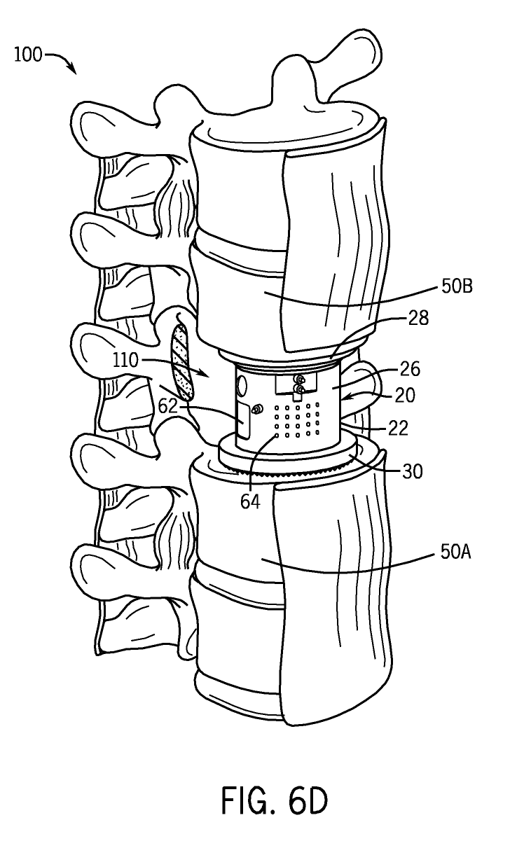

Referring now to FIG. 6A, a spine 100 includes a plurality of adjacent vertebrae, including an inferior vertebral body 50A, a superior vertebral body 50B, and a malignant vertebral body 50C disposed between and adjacent the vertebral bodies 50A and 50B. Disc material 102 separates the vertebral bodies. A tumor or metastasis 104 is shown at an outer surface of the vertebral body 50C, though it should be appreciated that the tumor 104 could be disposed inside the vertebral body 50C.

In accordance with one method, one or more brachytherapy seeds 24 can be implanted into the spine 100, either directly or carried by a spinal implant of the type described above or any alternatively constructed implant. Referring to FIG. 6B, a method for treating the spinal tumor 104 can include implanting the brachytherapy seeds 24 directly or percutaneously into or on the malignant vertebral body 50C, in addition to or as an alternative to implanting the brachytherapy seeds 24 to the adjacent vertebral bodies 50A-B or any alternative location on the spine 100. For instance, one of the outer end surface 73 of one or more brachytherapy seeds 24 can be spike-shaped in the manner described above, and driven directly into the various locations of the spine 100. The seeds 24 can be disposed entirely in the spine 100, or can protrude out from the spine 100.

Alternatively or additionally, one or more apertures 106 can be formed in the various locations of the spine 100, and a seed 24 can be implanted into each aperture 106. The seeds 24 can be press-fit in the apertures 106 without adhesive, or can be bonded to the aperture using a bone cement or alternative adhesive 108. The seeds 24 can be implanted prior to or subsequent to cement injection. Alternatively, the coupling element can be ultrasonically welded to the spine 100. Alternatively or additionally, the side surface 71 of one or more seeds 24 can be bonded directly to the spine 100, using an adhesive or ultrasonic welding.

Alternatively, the brachytherapy seeds 24 can be implanted indirectly into the spine 100, for instance via another spinal implant. In one embodiment, the spinal implant can include a screw 90 or nail 98 that carries one or more brachytherapy seeds 24 in the manner described above. The screw 90 and nail 98 can thus be driven into the spine 100 at locations as desired. The head 94 can be flush with the spine 100, or protrude out form the spine 100 as desired.

Referring now to FIG. 6C, the method of treating the tumor 104 can include removing the tumor 104 from the spine 100. In one embodiment, a corpectomy is performed whereby a portion or all of the vertebral body 50C that carries the tumor 104 is removed to define an intervertebral space 110 disposed between the adjacent vertebrae 100A and 100B. Alternatively or additionally, a hemicorpectomy is performed whereby a portion of a vertebral body is removed. In the illustrated embodiment, the surrounding disc material 102 has also been removed. It is appreciated that though the tumor 104 has been removed, it is common for malignant cells to remain in the spine 100, particularly if the tumor capsule is violated, which can allow malignant cells to escape and metastasize at various other locations in the spine 100. Thus, the seeds 24 can be implanted directly or indirectly to the vertebral endplates 51 of the adjacent vertebrae 50A and 50B in addition to the above-described spinal locations.

Referring to FIG. 6D, another method for indirectly implanting brachytherapy seeds in the spine 100 includes implanting the brachytherapy implant 20, or any alternative brachytherapy intervertebral implant, in the intervertebral space 110. The implant 22 can be provided having seeds 24 installed thereon or therein, or alternatively the seeds 24 can be installed in or on the implant 20 after the implant has been affixed in the intervertebral space 110. The seeds 24 can be disposed at any location on or in the implant 22 in orientations as described above suitable to direct radiation toward the likely locations that may contain one or more malignant cells. The expansion ring 36 can be manipulated as desired to bring the end plates 28 and 30 into engagement with the end plates of the vertebrae 50A and 50B.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the present description.

What is claimed is:

1. A brachytherapy spinal implant, comprising:
a spinal implant including a body extending along a longitudinal axis, a superior endplate disposed at one longitudinal end of the body, and an inferior endplate disposed at an opposing longitudinal end of the body, each end plate defining a vertebral-engaging surface configured to engage a complementary vertebral endplate; and
a plurality of irradiated seeds attached to the implant body so as to direct radiation toward the spine in a desired direction when the brachytherapy implant is disposed in an intervertebral space.

2. The brachytherapy implant as recited in claim 1, wherein the brachytherapy seeds are disposed in an aperture formed in the spinal implant.

3. The brachytherapy implant as recited in claim 2, wherein at least one of the apertures extends into the spinal implant at a depth that is less than a length of the brachytherapy seed disposed in the aperture.

4. The brachytherapy implant as recited in claim 2, wherein at least one of the apertures extends into the endplate at a depth that is less than a length of the brachytherapy seed disposed in the aperture, such that the brachytherapy seed protrudes out from the spinal implant.

5. The brachytherapy implant as recited in claim 2, wherein the brachytherapy seeds are press-fit into the aperture.

6. The brachytherapy implant as recited in claim 2, wherein the brachytherapy seeds are bonded to the spinal implant inside the aperture.

7. The brachytherapy implant as recited in claim 2, wherein the brachytherapy seeds are ultrasonically welded to the spinal implant inside the aperture.

8. The brachytherapy implant as recited in claim 2, wherein the aperture is formed in at least one of the endplates, such that the brachytherapy seed disposed in the aperture has an inferior or superior directional component.

9. The brachytherapy implant as recited in claim 8, wherein the brachytherapy seed provides a vertebral-engaging end.

10. The brachytherapy implant as recited in claim 2, wherein the aperture extends into the body.

11. The brachytherapy implant as recited in claim 10, wherein the aperture comprises a bone-packing aperture.

12. The brachytherapy implant as recited in claim 1, wherein the brachytherapy seeds define an elongate body presenting a side surface and a pair of opposing end surfaces, and the side surface is bonded a surface of the spinal implant.

13. The brachytherapy implant as recited in claim 12, wherein the spinal implant further comprises an endplate cap attached to the superior endplate, the endplate cap defining a base that is recessed with respect to the vertebral-engaging surface of the superior endplate, and the side surface of at least one of the brachytherapy seeds is attached to the base of the endplate cap.

14. A brachytherapy implant, comprising:
a shaft defining a proximal end and a distal end, and a head attached to the proximal end of the shaft, wherein the shaft defines a cannulation configured to receive one or more brachytherapy seeds.

15. The brachytherapy implant as recited in claim 14, wherein the shaft is threaded so as to provide a bone screw.

16. The brachytherapy implant as recited in claim 14, wherein the shaft is devoid of threads so as to provide a nail.

17. A method of treating a spinal tumor comprising the step of implanting a plurality of irradiated brachytherapy seeds into the spine at a location suitable to direct radiation toward the malignant cells.

18. The method as recited in claim 17, further comprising implanting the seeds percutaneously into the spine.

19. The method as recited in claim 17, further comprising implanting the seeds indirectly into the spine.

20. The method as recited in claim 19, wherein the seeds are disposed in a spinal implant, and the method further comprises the step of affixing the spinal implant to the spine.
